(19) United States
(12) Patent Application Publication
(10) Pub. No.: US 2017/0056332 A1
(43) Pub. Date: Mar. 2, 2017

(54) HEMORRHOID PREPARATION AND SHEETS

(71) Applicant: C.B. Fleet Company, Incorporated, Lynchburg, VA (US)

(72) Inventors: Nelson P. Ayala, Lynchburg, VA (US); Ping Qiu, Roanoke, VA (US); Debanjan Das, Lynchburg, VA (US)

(21) Appl. No.: 15/249,890

(22) Filed: Aug. 29, 2016

Related U.S. Application Data
(60) Provisional application No. 62/211,525, filed on Aug. 28, 2015.
(60) Provisional application No. 62/284,788, filed on Oct. 9, 2015.

(51) Int. Cl. A61K 9/70; A61K 31/135

(52) U.S. Cl. CPC A61K 9/70; A61K 31/135

(57) ABSTRACT

A pharmaceutical composition for the treatment of hemorrhoids, sheets, methods of making and using the same.

### BACKGROUND OF THE INVENTION

Field of the Invention

The present invention relates to pharmaceutical compositions for the treatment of hemorrhoids, sheets for applying the composition, methods of making and using the same.

Description of the Related Art

Hemorrhoids are swollen veins in the anal canal. Veins can swell inside the anal canal to form internal hemorrhoids or near the opening of the anus to form external hemorrhoids. Patients may have both. Excess pressure on the veins in the pelvic and rectal area can cause hemorrhoids. Normally, tissue inside the anus fills with blood to help control bowel movements. Increased pressure during bowel movements can cause the veins in this tissue to swell and stretch leading to hemorrhoids. Diarrhea or constipation also may lead to straining and can increase pressure on veins in the anal canal. Pregnant women can get hemorrhoids during the last 6 months of pregnancy because of increased pressure on the blood vessels in the pelvic area. Straining during labor can make hemorrhoids worse. Being overweight can also lead to hemorrhoids. WebMD Hemorrhoids Guide (2015).

Common symptoms of both internal and external hemorrhoids include: bleeding during bowel movements, discomfort, skin irritation, itching, rectal pain. Internal hemorrhoids often are small, swollen veins in the wall of the anal canal. External hemorrhoids can get irritated and clot under the skin, causing a hard painful lump called a thrombosed, or clotted, hemorrhoid.

Current medicines that can help relieve symptoms of hemorrhoids include ointments, wipes, suppositories, and nonprescription pain relievers. WebMD Hemorrhoids Guide (2015).

Ointments that protect the skin, such as zinc oxide or petroleum jelly, can prevent further injury and reduce itching by forming a barrier over hemorrhoids. Medicated ointments may contain 1% or 2.5% hydrocortisone that may relieve inflammation and itching. Other products contain a local anesthetic that numbs the anal region for relief of pain associated with hemorrhoids. WebMD Hemorrhoids Guide (2015).

Suppositories. Preparation H® or Tucks® (formerly Anusol), may last for 7 to 10 days to relieve irritation and to lubricate the anal canal during bowel movements. Some of these products contain substances that can harm anal tissues if they are used for too long. WebMD Hemorrhoids Guide (2015).

Nonprescription pain relievers such as Tylenol® (acetaminophen) can help with pain. Aspirin and other nonsteroidal anti-inflammatory drugs (NSAIDs) such as Motrin® (ibuprofen) and Aleve® (naproxen) can offer patients relief from pain and swelling. WebMD Hemorrhoids Guide (2015).

Commercially-available products for the treatment of hemorrhoids include Preparation H® Wipes which have 50% Witch Hazel (astringent) as an active ingredient and aloe barbadensis leaf juice, anhydrous citric acid, capryl/capramidopropyl betaine, diazolidinyl urea, glycerin, methyl paraben, propylene glycol, propyl paraben, purified water, and sodium citrate as inactive ingredients. Similarly. Preparation H® Medicated Wipes for Women which have 20% Witch Hazel (astringent) as an active ingredient and aloe barbadensis leaf juice, anhydrous citric acid, capryl/capramidopropyl betaine, chamomilla recutita (matricaria) flower extract. cucumis sativus (cucumber) fruit extract, diazolidinyl urea, dipropylene glycol. DMDM hydantoin, edetate disodium, fragrance, glycerin, methylisothiaizolinone, methyl paraben. PEG-75 shea butter glycerides, phenoxyethanol, polysorbate 20 (Polyoxyethylene (20) sorbitan monolaurate), propylene glycol, propyl paraben, purified water, sodium citrate, and vitamin E acetate as inactive ingredients. Preparation H® Maximum Strength Pain Relief Cream which comprises 14.4% glycerin, 0.25% phenylephrine HCl, 1% pramoxine HCl, and 15% white petrolatum. Preparation H® Wipes; Preparation H® Medicated Wipes for Women; and Preparation H® Maximum Strength Pain Relief Cream product information (2015).

Tucks® Medicated Cooling pads comprise 50% Witch Hazel (astringent) as an active ingredient and water, glycerin, alcohol, propylene glycol, sodium citrate, diazolidinyl urea, citric acid, methyl paraben, and propyl paraben as inactive ingredients. Tucks® Medicated Cooling pads product information (2015).

Nelsons Medicated Wipes comprise Aesculus Hippocastanum-6 C, Calendula Officinalis-6 C. Collinsonia Canadensis-6 C. Hamamelis Virginiana-6 C, Paeonia Officinalis-6 C as active ingredients and aloe vera, butylparaben, citric acid, diazolidinyl urea, disodium cocoamphodiacetate, ethylparaben, glycerin, isobutylparaben, methyl paraben, phenoxyethanol, polysorbate 20, propylene glycol, propyl paraben, purified water, sodium hydroxide, and tocopherol as inactive ingredients. Nelsons Medicated Wipes product information (2015).

CVS® Maximum Strength Formula Medicated wipes comprise 50% Witch Hazel (astringent) as an active ingredient and aloe barbadensis leaf juice, 2-bromo-2-nitropropane-1,3-diol, citric acid, disodium cocoamphodiacetate, glycerin, iodopropynyl butylcarbamate, PEG-75 lanolin, propylene glycol, sodium citrate, and water as inactive ingredients. CVS Pharmacy website (2015).

Walgreens® Pre-Moistened Medicated Wipes comprise 50% Witch Hazel (astringent) as an active ingredient and alcohol, citric acid, glycerin, phenoxyethanol, potassium sorbate, purified water, and sodium citrate as inactive ingredients. Walgreens Website (2015).

The commercially available preparations may not provide sufficient relief to patients with hemorrhoids. Accordingly, there is a need in the art for an improved hemorrhoid treatment preparation.

### BRIEF SUMMARY OF THE INVENTION

In numerous embodiments, this invention provides a pharmaceutical composition comprising an analgesic, vasoconstrictor, emollient, and cooling agent.

In one embodiment, the analgesic may be pramoxine hydrochloride, benzocaine; benzyl alcohol, dibucaine, dibucaine hydrochloride, dyclonine hydrochloride, lidocaine, pramoxine hydrochloride, tetracaine, tetracaine hydrochloride, or a mixture thereof.

In most embodiments of this invention, the analgesic is pramoxine hydrochloride. In numerous embodiments of this invention, the analgesic may be in an amount of about 0.5% to about 2.0% by weight of the composition. In various embodiments of this invention, the analgesic may be in an amount of about 0.75%, 1.0%, 1.25%, or 1.50% by weight of the composition. In most embodiments of this invention, the analgesic may be benzocaine at about 5 to 20 percent by weight; benzyl alcohol at about 1 to 4% by weight; dibucaine at about 0.25 to 1% by weight; dibucaine hydrochloride at about 0.25 to 1% by weight; dyclonine hydrochloride at about 0.5 to 1% by weight; lidocaine at about 2 to 5% by weight; pramoxine hydrochloride at about 1% by weight; tetracaine at about 0.5 to 1% by weight; tetracaine hydrochloride at about 0.5 to 1%; or a mixture thereof.

The compositions of this invention may comprise water as a solvent.

In most embodiments of this invention, the composition may comprise mixtures of water, glycerin, propylene glycol, and/or low molecular weight polyethylene glycols (PEGs) as a solvent. In many embodiments, the composition may comprise about 10-40% glycerin by weight, preferably 10%, 15%, 20%, 25%, 30%, 35%, or 40% glycerin by weight, more preferably about 20% glycerin by weight. In most embodiments, the composition may comprise about 1-10% propylene glycol by weight, preferably about 1%, 1.5%, 2%, 2.5%, 3%, 5%, 6%, 7%, 8%, 9%, or 10% propylene glycol by weight, more preferably about 2% propylene glycol by weight. In particular embodiments, the composition may comprise about 1-5% polyethylene glycol by weight, preferably about 1%, 1.5%, 2%, 2.5%, 3%, 3.5%, 4%, 4.5%, or 5% polyethylene glycol by weight, more preferably about 2% polyethylene glycol by weight.

In some embodiments, the said polyethylene glycol may have an average molecular weight of 100-600. In many embodiments, the polyethylene glycol may be PEG100, PEG200, PEG300, PEG400, PEG500, PEG600 or a mixture thereof. In many embodiments, the polyethylene glycol may be PEG400, PEG600, or a mixture thereof. In many embodiments, the composition may comprise a mixture of water:propylene glycol:PEG in a ratio of 1:1:1 to 3:2:1. In many embodiments, the composition may comprise a mixture of water:propylene glycol:PEG100 in a ratio of 1:1:1. In many embodiments, the composition may comprise a mixture of water:propylene glycol:PEG600 in a ratio of 3:2:1.

In particular embodiments, the solvent ma, be in an amount of about 70% to about 90% by weight of the composition. In particular embodiments, the solvent may be in an amount of about 71%, 72%, 73%, 74%, or 75% by weight of the composition.

In most embodiments of this invention, the vasoconstrictor may be ephedrine sulfate, epinephrine, epinephrine hydrochloride, phenylephrine hydrochloride, or a mixture thereof. In particular embodiments, the vasoconstrictor may be phenylephrine hydrochloride. In many embodiments, the vasoconstrictor may be in an amount of about 0.005% to about 1.25% by weight of the composition. In particular embodiments, the vasoconstrictor may be in an amount of about 0.20%, 0.25%, 0.30%, or 0.35% by weight of the composition. In particular embodiments, the vasoconstrictor may be an amount of about 0.1 to 1.25% by weight. In particular embodiments, the vasoconstrictor may be ephedrine sulfate at about 0.1 to 1.25% by weight; epinephrine at about 0.005 to 0.01% by weight; epinephrine hydrochloride at about 0.005 to 0.01% by weight; phenylephrine hydrochloride at about 0.25%; or a mixture thereof. In another embodiment, the vasoconstrictor may be epinephrine in an amount of about 0.10% to about 0.50% by weight of the composition.

In many embodiments, the emollient may be glycerin, propylene glycol, dipropylene glycol, hexylene glycol, butylene glycol, or a combination thereof. In many embodiments, the emollient may be propylene glycol. In many embodiments, the emollient may be glycerin. In many embodiments, the emollient may be in an amount of about 1.0% to about 40% by weight of the composition. In particular embodiments, the emollient may be in an amount of about 2.0%, 5.0%, 10%, 15%, 20%, 25%, 30%, 35%, or 40% by weight of the composition.

In many embodiments, the composition may comprise a surfactant which is preferably a nonionic surfactant. In many embodiments, the nonionic surfactant may be ethoxylated castor oil, Polysorbate 20, Polysorbate 60, Polysorbate 80, or a combination thereof. In many embodiments, the composition may comprise a surfactant which is Polysorbate 80 (polyethylene glycol sorbitan monooleate).

In many embodiments, the surfactant may be in an amount of about 0.5% to about 5% by weight of the composition. In particular embodiments, the surfactant may be in an amount of about 1.0%, 1.5%, 2.0%, 3.0%, 3.5%, or 5.0% by weight of the composition.

In many embodiments, the cooling agent may be menthol, eucalyptol, camphor, juniper tar, or a combination thereof. In many embodiments, the cooling agent may be menthol. In many embodiments, the cooling agent may be in an amount of about 0.05% to about 0.1% by weight of the composition. In particular embodiments, the cooling agent may be in an amount of about 0.05%, 0.06%, 0.07%, 0.08%, or 0.09% by weight of the composition.

In many embodiments, the composition may further comprise an antioxidant, acidulant, preservative, chelating agent, or a combination thereof.

In many embodiments, the antioxidant may comprise butylated hydroxyanisole, butylated hydroxyl toluene, propyl gallate, or a combination thereof. In many embodiments, the antioxidant may be in an amount about 0.01% to about 0.10% by weight of composition. In particular embodiments, the antioxidant may be in an amount about 0.03%, 0.04%, 0.05%, 0.06%, or 0.07% by weight of composition.

In many embodiments, the acidulant may be sodium citrate, citric acid, or a combination thereof. In many embodiments, the acidulant may be in an amount about 0.10% to about 0.50% by weight of composition. In particular embodiments, the acidulant may be in an amount about 0.20%, 0.25%, 0.30%, 0.35%, or 0.40% by weight of composition.

In many embodiments, the preservative may be sodium benzoate, methyl paraben, propyl paraben, or a combination thereof, in many embodiments, the preservative may be is in an amount about 0.01% to about 0.25% by weight of composition. In particular embodiments, the preservative may be in an amount about 0.01%, 0.02%, 0.03%, 0.05%, 0.10%, 0.15%, 0.16%, 0.18%, 0.20%, or 0.25% by weight of composition.

In many embodiments, the chelating agent may be disodium EDTA. In many embodiments, the chelating agent may be in an amount about 0.05% to about 0.20% by weight of composition. In particular embodiments, the chelating agent may be in an amount about 0.08%, 0.09%, 0.10%, 0.11%, 0.12%, or 0.13% by weight of composition.

In many embodiments of the method of this invention, the pharmaceutical composition described herein will not contain petrolatum.

In many embodiments of the method of this invention, the pharmaceutical composition described herein will not contain witch hazel.

In many embodiments of the method of this invention, the pharmaceutical composition described herein will not contain cetyl alcohol.

In many embodiments of the method of this invention, the pharmaceutical composition described herein may be formulated as a suspension.

In a further embodiment, the invention provides for a pharmaceutical composition comprising a solvent, emollient, analgesic, vasoconstrictor, antioxidant, acidulant, surfactant, preservative, chelating agent, and a cooling agent/penetrating agent. In another embodiment, the pharmaceutical composition may comprise water, glycerin, pramoxine hydrochloride, phenylephrine hydrochloride, propylene glycol, butylated hydroxyanisole, trisodium citrate dihydrate, TWEEN® 80 (Polysorbate 80), citric acid (anhydrous), sodium benzoate, disodium EDTA, methyl paraben, propyl paraben, and L-menthol crystals. In a particular embodiment, the pharmaceutical composition may comprise about 73% water by weight, 20% glycerin by weight, 1% pramoxine hydrochloride by weight, 0.25% phenylephrine hydrochloride by weight, 2% propylene glycol by weight, 0.05% butylated hydroxyanisole by weight, 0.35% trisodium citrate dihydrate by weight, 2% TWEEN® 80 (Polysorbate 80) by weight, 0.25% citric acid (anhydrous) by weight, 0.1% sodium benzoate by weight, 0.1% disodium EDTA by weight, 0.18% methyl paraben by weight, 0.02% propyl paraben by weight, and 0.07% L-menthol crystals by weight.

In many embodiments of this invention, a cream may comprise the pharmaceutical composition described herein. In many embodiments of this invention, a lotion may comprise the pharmaceutical composition described herein.

In many embodiments of this invention, a sheet may comprise the pharmaceutical composition described herein. In certain embodiments, the sheet may comprise absorbent cellulose based fibers, absorbent synthetic fibers, or a mixture thereof. The fibers may be cross-linked. The absorbent cellulosic fiber may be in sheet form, fluff form, or a combination thereof. The absorbent cellulose fiber may be in the form of a stabilized resin-bonded or thermal-bonded non-woven mat. The absorbent cellulose fiber may be a wood pulp fiber obtained from a Kraft or sulfite chemical process. The wood pulp fiber may be obtained from a hardwood cellulose pulp, a softwood cellulose pulp, or a combination or mixture thereof. The composition may be applied to the sheet to provide from about 0.001 weight % to about 600% weight % composition on fiber, based on the total weight of the fiber. The composition may be applied to the sheet to provide from about 6.0 gram solution per sheet of cloth.

In many embodiments of this invention, a method of making a sheet may comprise applying the pharmaceutical composition described herein to absorbent fibers. The composition may be applied by spraying, dipping, or rolling. The composition may be applied by a puddle press, size press, or a blade-coater.

In many embodiments of this invention, a method of making the pharmaceutical composition described herein may comprise (a) mix BHA, polysorbate 80, and propylene glycol for form a first mixture; (b) add methyl paraben, propyl paraben, and menthol to said first mixture to form a second mixture; (c) mix water and glycerin to form a third mixture; (d) add EDTA, sodium benzoate, sodium citrate, and citric acid to said third mixture to form a fourth mixture; (e) add pramoxine HCl and phenylephrine HCl to said fourth mixture to form a fifth mixture; and (f) add said second mixture to said fifth mixture to form a hemorrhoid treatment composition.

In many embodiments of this invention, a method of treating hemorrhoids may comprise administering one or another embodiment of the pharmaceutical composition described herein a patient in need thereof. The composition is applied by the patient to the source of the pain, redness or swelling. This area of application may be the hemorrhoid itself or the tissue adjacent to the hemorrhoid.

In many embodiments of this invention, a method of treating hemorrhoids comprises applying the sheet described herein to a hemorrhoid on patient in need thereof.

In one embodiment, a composition for treating hemorrhoids may comprise analgesic, vasoconstrictor, and a cooling agent.

In one embodiment, the use of a composition comprising analgesic, solvent, vasoconstrictor, emollient, surfactant, and cooling agent, may be used in the manufacture of a composition for treating hemorrhoids.

### DESCRIPTION OF THE DRAWINGS

FIG. 1 depicts an exemplary flow chart of manufacturing hemorrhoid treatment compositions.

### DETAILED DESCRIPTION OF EXEMPLARY EMBODIMENTS

Commercially available hemorrhoid treatment preparations provide partial relief to hemorrhoid sufferers. Further, commercially available hemorrhoid treatment preparations have lipophilic skin protectants which delay the action of the anorectal drugs. Specifically, the addition of oleaginous additives such as shark liver oil, garlic oil, petrolatum, mineral oil, stearyl alcohol, cetyl alcohol, and tocopherol have occlusive properties that delay the onset of water soluble drugs and are often uncomfortable for patients to use due to greasiness.

The hemorrhoid treatment compositions described herein comprise multiple ingredients, which may include analgesic, vasoconstrictor, hydrophilic skin protectant, penetration enhancer, especially hydrophilic penetration enhancer, surfactant, and cooling agent. The hemorrhoid treatment compositions described herein may comprise about 1% pramoxine HCl (analgesic), 0.25% phenylephrine HCl (vasoconstrictor), 20% glycerin (hydrophilic skin protectant), 2% propylene glycol (hydrophilic penetration enhancer), 2% TWEEN® 80 (polyethylene glycol sorbitan monooleate), 0.07% D,L-menthol (cooling agent/penetration agent), and water to balance. See, e.g., Table 1.

The inherent solubility parameter of skin lipids (δs) is about 10 (cal/cm³)^{1/2}, where water the δv is 23.4. The inherent solubility parameter for lipophilic ingredients of prior art preparations include shark liver oil/squalene (δv=6.19), petrolatum (δv=7.3), mineral oil (δv=7.09), stearyl alcohol (δv=8.90), and cetyl alcohol (δv=8.94) which are added to provide skin protection. These lipophilic skin protectants have occlusive properties that can act to delay the onset of action for water soluble drugs, especially when the inflamed area is located deep inside the tortuous and swollen hemorrhoid tissue. In contrast to the skin protectants of the prior art, propylene glycol (δv=14.5) and glycerin (δv=16.26) are capable of distributing within the stratum corneum and increasing the flux (enhancing penetration) of water soluble drugs, thereby acting as penetration enhancers. Menthol (δv=9.94) is capable of distributing with the stratum corneum and increasing the flux of water soluble drugs.

A single agent may act as both cooling agent and penetration enhancer, e.g., menthol. Obata, et al. reported that percutaneous absorption of hydrophilic diclofenac sodium was substantially enhanced in the presence of I-menthol. Obata, et al. Drug Design. Del., 6, 319, 1990. The combined effect of menthol and ethanol as skin penetration enhancers was also studied by Kobayashi, et al. Pharm Res., 11, 96, 1994.

The inventors surprisingly discovered that menthol acts synergistically both as a cooling agent and a penetration enhancer to improve the delivery of %% ater soluble active ingredient through the mucous membrane of a hemorrhoid. The menthol is preferably solubilized in a micro-emulsion of a surfactant, e.g. TWEEN® 80 (polyethylene glycol sorbitan monooleate). Other surfactants that may be used are nonionic surfactants such as ethoxylated castor oil. Polysorbate 20 (Polyoxyethylene (20) sorbitan monolaurate), and/or Polysorbate 60 (polyoxyethylene (60) sorbitan monostearate). The hemorrhoid treatment compositions described herein improve the efficacy of the hemorrhoid preparation through the synergistic action of analgesic (e.g., pramoxine HCl), vasoconstrictor (e.g., phenylephrine HCl) and penetration enhancer (e.g., methanol, propylene glycol, and glycerin). Further, the hemorrhoid treatment compositions described herein do not contain lipophilic skin protectants that slow down the action of the water soluble drugs.

The pharmaceutical compositions described herein may comprise water, glycerin, propylene glycol, and/or low molecular weight polyethylene glycols (PEGs) as a solvent. For example, the pharmaceutical compositions described herein may comprise about 10-40% glycerin by weight. The pharmaceutical compositions described herein may comprise, 10%, 15%, 20%, 25%, 30%, 35%, or 40% glycerin by weight, preferably about 20% glycerin by weight. The pharmaceutical compositions described herein may comprise about 1-10% propylene glycol by weight. The pharmaceutical compositions described herein may comprise, about 1%, 1.5%, 2%, 2.5%, 3%, 5%, 6%, 7%, 8%, 9%, or 10% propylene glycol by weight, preferably about 2% propylene glycol by weight. The pharmaceutical compositions described herein may comprise about 1-5% polyethylene glycol, preferably PEG400, by weight. The pharmaceutical compositions described herein may comprise, about 1%, 1.5%, 2%, 2.5%, 3%, 3.5%, 4%, 4.5%, or 5% polyethylene glycol, preferably PEG400, by weight, preferably about 2% polyethylene glycol, preferably PEG400, by weight.

Analgesics that may be used in compositions of this invention include but are not limited to benzocaine; benzyl alcohol; dibucaine; dibucaine hydrochloride; dyclonine hydrochloride; lidocaine; pramoxine hydrochloride; tetracaine; tetracaine hydrochloride; or a mixture thereof.

A preferred analgesic is pramoxine HCl.

The analgesics may be in an amount of about 0.25 to 20% by weight. For example, benzocaine may be at about 5 to 20% by weight; benzyl alcohol may be at about 1 to 4% by weight; dibucaine may be at about 0.25 to 1% by weight; dibucaine hydrochloride may be at about 0.25 to 1% by weight; dyclonine hydrochloride may be at about 0.5 to 1% by weight; lidocaine may be at about 2 to 5% by weight; pramoxine hydrochloride may be at about 1 percent by weight; tetracaine may be at about 0.5 to 1% by weight; tetracaine hydrochloride may be at about 0.5 to 1%; or a mixture thereof.

The A preferred analgesic is pramoxine HCl may be in an amount of about 0.5% to about 2.0% by weight of the composition. For example, pramoxine HCl may be in an amount of about 0.75%, 1.0%, 1.25%, or 1.50% by weight of the composition.

Vasoconstrictors used in compositions of this invention include ephedrine sulfate, epinephrine, epinephrine hydrochloride, phenylephrine hydrochloride, or a mixture thereof. The vasoconstrictor may be present in an amount of about 0.005 to 1.25% by weight. The vasoconstrictor may be ephedrine sulfate at about 0.1 to 1.25% by weight; epinephrine at about 0.005 to 0.01% by weight; epinephrine hydrochloride at about 0.005 to 0.01% by weight; phenylephrine hydrochloride at about 0.25%; or a mixture thereof.

A preferred vasoconstrictor is phenylephrine HCl. The preferred vasoconstrictor phenylephrine HCl may be in an amount of about 0.10% to about 0.50% by weight of the composition. For example, the compositions described herein may comprise phenylephrine HCl in an amount of about 0.20%, 0.25%, 0.30%, or 0.35% by weight of the composition.

The compositions of this invention may comprise an emollient.

Preferred emollients are polyhydric alcohols that dry without leaving a sticky residue. Examples of suitable emollients are dipropylene glycol, hexylene glycol, butylene glycol which are used at percentages low enough to ensure solubility in the preparation.

The emollient may be propylene glycol. The emollient may be glycerin.

The emollient may be in an amount of about 1.0% to about 40% by weight of the composition. The emollient may be in an amount of about 2.0%, 5.0%, 10%, 15%, 20%, 25%, 30%, 35%, or 40% by weight of the composition. For example, the composition may comprise about 1-40% glycerin by weight of the composition, preferably, about 2.0%, 5.0%, 10%, 15%, 20%, 25%, 30%, 35%, or 40% by weight of the composition. The composition may comprise about 1-40% propylene glycol by weight of the composition, preferably about 2.0%, 5.0%, 10%, 15%, 20%, 25%, 30%, 35%, or 40% by weight of the composition.

The compositions described herein may further comprise an antioxidant, acidulant, preservative, chelating agent, or a combination thereof.

The compositions of this invention will typically comprise acidulants, such as citric acid and sodium citrate, as well as preservatives, such as methyl paraben, propyl paraben, sodium benzoate, diazolidinyl urea, and butylated hydroxyanisole. The compositions may also include chelating agents. Compositions of this invention also comprise an aqueous solvent.

The antioxidant may be butylated hydroxyanisole, butylated hydroxyl toluene, propyl gallate, or a combination thereof. The antioxidant, preferably butylated hydroxyanisole, may be in an amount about 0.01% to about 0.10% by weight of composition, preferably about 0.03%, 0.04%, 0.05%, 0.06%, or 0.07% by weight of composition.

The acidulant may be sodium citrate, citric acid, or a combination thereof.

The acidulant may be in an amount about 0.10% to about 0.50% by weight of composition, preferably in an amount about 0.20%, 0.25%, 0.30%, 0.35%, or 0.40% by weight of composition.

For example, the composition may comprise water as a solvent. The composition may comprise a mixture of water/glycerin/propylene glycol/low molecular weight polyethylene glycols (PEGs) as a solvent. The composition may comprise a solvent is in an amount of about 70% to about 90% by weight of the composition. For example, the composition may comprise a solvent, preferably water, that is in an amount of about 71%, 72%, 73%, 74%, or 75% by weight of the composition.

The pharmaceutical composition may comprise water as a solvent. The may be in an amount of about 70% to about 90% by weight of the composition. For example, the solvent may be in an amount of about 71%, 72%, 73%, 74%, or 75% by weight of the composition.

The pharmaceutical composition may comprise mixtures of water, glycerin, propylene glycol, and/or low molecular weight polyethylene glycols (PEGs) as a solvent. The pharmaceutical composition may comprise about 10-40% glycerin by weight, preferably 10%, 15%, 20%, 25%, 30%, 35%, or 40% glycerin by weight, more preferably about 20% glycerin by weight. The pharmaceutical composition may comprise about 1-10% propylene glycol by weight, preferably about 1%, 1.5%, 2%, 2.5%, 3%, 5%, 6%, 7%, 8%, 9%, or 10% propylene glycol by weight, more preferably about 2% propylene glycol by weight. The pharmaceutical composition may comprise about 1-5% polyethylene glycol by weight, preferably about 1%, 1.5%, 2%, 2.5%, 3%, 3.5%, 4%, 4.5%, or 5% polyethylene glycol by weight, more preferably about 2% polyethylene glycol by weight.

The polyethylene glycol may have an average molecular weight of 100-600. The polyethylene glycol may be PEG100, PEG200, PEG300, PEG400, PEG500, PEG600 or a mixture thereof. The polyethylene glycol may be PEG400, PEG600, or a mixture thereof. Ratio of the mixtures depend on the average molecular weight of PEG where the number denotes the molecular unit, e.g. PEG400 has an average molecular weight of 400. In the compositions described herein, water soluble PEGs can vary from PEG100 to PEG600. As the molecular weight of the PEG increases, the proportion of the PEG in the mixture may decrease. For example, the ratio of a suitable mixture of water:propylene glycol:PEG100=1:1:1 and the ratio of a suitable mixture of water:propylene glycol:PEG600=3:2:1 The pharmaceutical composition may comprise a mixture of water:propylene glycol:PEG in a ratio of 1:1:1 to 3:2:1. For example, the water:propylene glycol:PEG100 may be a ratio of 1:1:1. The water:propylene glycol:PEG600 may be a ratio of 3:2:1.

The solvent may be in an amount of about 70% to about 90% by weight of the composition. For example, the solvent may be in an amount of about 71%, 72%, 73%, 74%, or 75% by weight of the composition.

The compositions described herein may comprise a nonionic surfactant. The nonionic surfactant may be ethoxylated castor oil. Polysorbate 20. Polysorbate 60. Polysorbate 80, or a combination thereof. The surfactant may be Polysorbate 80 (polyethylene glycol sorbitan monooleate). The surfactant, preferably Polysorbate 80 (polyethylene glycol sorbitan monooleate), may be in an amount of about 0.5% to about 5% by weight of the composition, preferably in an amount of about 1.0%, 1.5%, 2.0%, 3.0%, 3.5%, or 5.0% by weight of the composition.

Cooling agents used in the compositions of this invention include menthol, eucalyptol, camphor, juniper tar, or a combination thereof. A preferred cooling agent is menthol.

The cooling agent, preferably menthol, may be an amount of about 0.05% to about 0.1% by weight of the composition, preferably about 0.05%, 0.06%, 0.07%, 0.08%, or 0.09% by weight of the composition.

The preservative is a pharmaceutically-acceptable preservative suitable for topical use. For example, the preservative may be sodium benzoate, methyl paraben, propyl paraben, or a combination thereof. The preservative may be in an amount about 0.01% to about 0.25% by weight of composition. For example, the preservative may be in an amount about 0.01%, 0.02%, 0.03%, 0.05%, 0.10%, 0.15%, 0.16%, 0.18%, 0.20%, or 0.25% by weight of composition.

The chelating agent may be disodium EDTA in an amount about 0.05% to about 0.20% by weight of composition, preferably in amount about 0.08%, 0.09%, 0.10%, 0.11%, 0.12%, or 0.13% by weight of composition.

The compositions described herein do not contain petrolatum. The compositions described herein do not contain witch hazel. The compositions described herein do not contain acyclic alcohol or a monohydric aliphatic alcohol. The compositions described herein do not contain cetyl alcohol. The compositions described herein may be formulated as a suspension, cream, lotion, or applied to a sheet.

The hemorrhoid treatment compositions described herein have rapid onset action because, in part, it is a solution and not a cream which reaches the affected areas faster than a petrolatum-containing cream. Also, the hemorrhoid treatment compositions described herein contain menthol which has instant cooling action that provides the patient rapid relief from pain associated with hemorrhoids. Also, the hemorrhoid treatment compositions described herein comprise a penetration enhancer—menthol—that allows for better access of the active ingredients to the inflamed tissue. The hemorrhoid treatment compositions described herein use glycerin (e.g., 20%) and not petrolatum which gives the compositions better skin protectant effect. The hemorrhoid treatment compositions described herein has a rapid synergistic effect because the active agents are solubilized in a solution for rapid onset of action. Also, menthol has a dual penetration enhancer/cooling agent that causes the active agents to penetrate the affected area and provide long lasting relief from the pain and discomfort of hemorrhoids. Menthol, a penetration agent, improves the analgesic action of the pramoxine HCl as a topical analgesic and phenylephrine HCl as a topical vasoconstrictor. The improvement in action is related to the fact that neutral molecules such as menthol have the exact solubility characteristics that improve the passage of ionic molecules through mucous membranes.

Also, the hemorrhoid treatment compositions described herein do not contain any petrolatum and are non-greasy with a better feel after application. Additionally, the microemulsion created by the addition of at least 2% w/w TWEEN® 80 forms micelles that solubilize menthol.

A clear microemulsion is inherently stable due to the formation of submicron sized micelles that are also referred to as isotropic solutions. The surfactants which make up microemulsions are too large themselves to penetrate into the dermis.

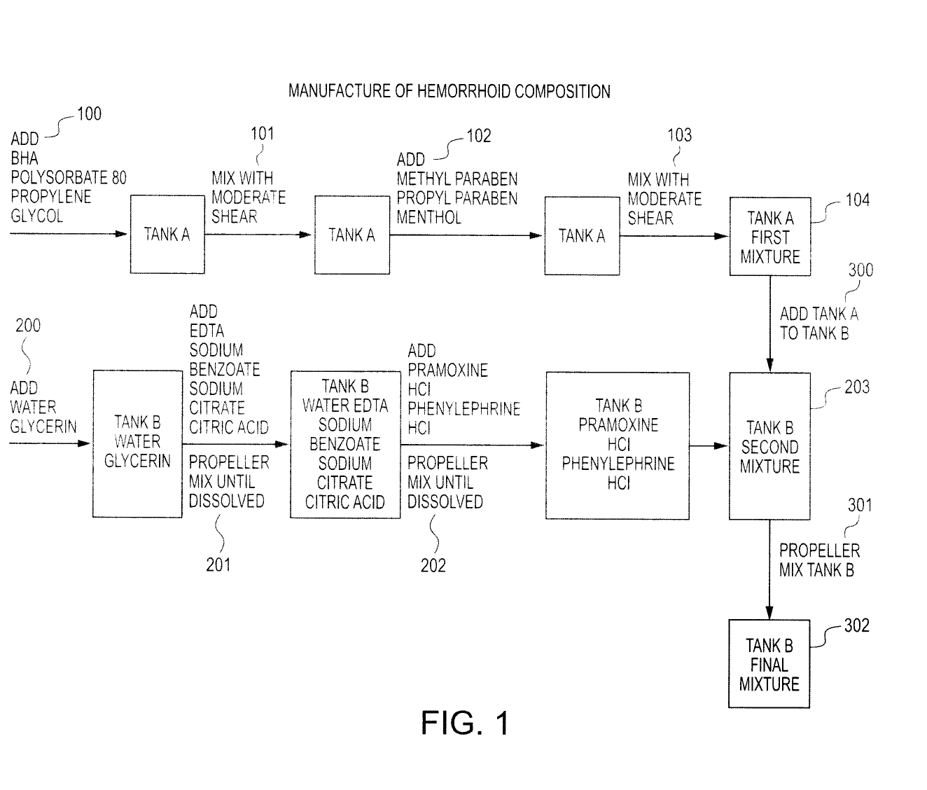

The hemorrhoid treatment preparations described herein may be formulated as a suppository, topical solution, ointment, cream, or impregnated into a wipe. Further, the hemorrhoid treatment preparations described herein may be delivered by a pump spray, squeeze tube (with or without nozzles), or disposable applicators. The dose may be metered by the pump spray or disposable applicator. In certain embodiments, the composition of this invention may be produced by mixing menthol with surfactant(s) and preservatives, then adding this mixture to an aqueous solution which contains all of the other ingredients of the composition. FIG. 1 shows an exemplary manufacturing scheme. This final mixture may then be impregnated into a non-woven sheet for use as a wipe.

A sheet may be impregnated with the composition described herein. The sheet may be absorbent cellulose based fibers, absorbent synthetic fibers, or a mixture thereof. Further, the fibers of the sheet may be cross-linked. The cellulosic absorbent fiber may be in sheet form, fluff form, or a combination thereof. The absorbent cellulose fiber is may be in the form of a stabilized resin-bonded or thermal-bonded non-woven mat. The absorbent cellulose fiber may be a wood pulp fiber obtained from a Kraft or sulfite chemical process. The wood pulp fiber may be obtained from a hardwood cellulose pulp, a softwood cellulose pulp, or a combination or mixture thereof. The sheet may be impregnated with the compositions described herein by dipping, spraying, rolling, or a combination thereof.

Preferred cloths are those which are dispersible. Dispersibility is a desirable characteristic for cloths due to the concern about the potential blockage of sewer drains and septic systems. Specific tests proposed by the National Sanitary Association are performed in order for a cloth to be designated as “dispersible”.

Proceeding now to a description of the drawing. FIG. 1 shows an exemplary method of manufacturing the composition described herein. The composition is formulated as two separate formulations 104, 203 and then mixed together into a final composition 302. BHA is added to polysorbate 80 and propylene glycol 100. This composition is mixed with moderate shear 101. Then methyl paraben, propyl paraben, and menthol are added 102. This mixture is then mixed with moderate shear 103 to form a first mixture 104.

The amount of shear should be sufficient to create a vortex in the solution such that the BHA, methyl paraben, propyl paraben, and menthol will be dissolved in a reasonable period of time, preferably less than 1 hour. This amount of mixing can be achieved with a 6″-12″ propeller mixer running at 600-1200 RPM. For example, in a 100 gallon tank a 6″ blade running at 600 RPM may be used to achieve dissolution in about 1 hour. As the size of the tank increases, the mixer speed and/or the size of the blade may need to be increased in order to achieve adequate mixing in a reasonable amount of time, preferably less than 1 hour. For example, the mixer speed may be increased to 1200 RPM and the size of the blade may be increased to 12″ in order to achieve dissolution in about 1 hour.

Ross, Silverson, or Gaulin mixers may be used. For example, moderate shear may be 400 rpm (rotations per minute) of the 4 blade rotor head against a static stator with a 10 micron gap allowing the mixture to pass through in a Ross® High Shear Mixer (Laboratory Model). The time required for mixing will depend upon the size of the batch and the number of passes through the mixer head in one hour, usually at least 3-5 passes per hour are required to achieve dissolution. Slight heating will accelerate the mixing process, but heat is not required to achieve mixing.

In a separate tank, water and glycerin are added together 200. Then EDTA, sodium benzoate, sodium citrate, citric acid are added and propeller mixed until dissolved 201. Then pramoxine HCl, phenylephrine HCl are added and the mixture is propeller-mixed until dissolved 202. This produces a second mixture 203. Then the first mixture 104 is added to the second mixture 203 and propeller mixed in the reaction vessel that held the second mixture 301. This produces the final formulation 302, a composition for treating hemorrhoids.

Further embodiments of the present invention will now be described with reference to the following examples. The examples contained herein are offered by way of illustration and not by any way of limitation.

### Example 1

TABLE 1
Non-optimal hemorrhoid treatment preparation

| Ingredient | Ingredient Function | % w/w |
|---|---|---|
| water | solvent | 51.70% |
| glycerin | emollient | 42.61% |
| pramoxine hydrochloride | analgesic | 1.00% |
| phenylephrine hydrochloride | vasoconstrictor | 0.25% |
| propylene glycol | emollient | 2.00% |
| butylated hydroxyanisole | antioxidant | 0.05% |
| trisodium citrate dihydrate | acidulant | 0.35% |
| TWEEN ® 80 | surfactant | 1.30% |
| (Polysorbate 80) | | |
| citric acid (anhydrous) | acidulant | 0.25% |
| sodium benzoate | preservative | 0.10% |
| disodium EDTA | chelating agent | 0.10% |
| Methyl paraben | preservative | 0.18% |
| Propyl paraben | preservative | 0.02% |
| Eucalyptol | cooling agent/ | 0.02% |
| | penetrating agent | |
| L-menthol crystals | cooling agent/ | 0.07% |
| | penetrating agent | |

Clear microemulsions are inherently stable solutions. The preparation shown in Table 1 was made with 1.3% w/w TWEEN® 80 and was not clear, therefore the microemulsion was not completely formed. Some of the menthol and eucalyptol remained undissolved. Also, is preparation was found to be sticky while drying on the skin.

### Example 2

TABLE 2
Non-optimal hemorrhoid treatment preparation

| Ingredient | Ingredient Function | % w/w |
|---|---|---|
| water | solvent | 74.11% |
| glycerin | emollient | 20.00% |
| pramoxine hydrochloride | analgesic | 1.00% |
| phenylephrine hydrochloride | vasoconstrictor | 0.25% |
| propylene glycol | emollient | 2.00% |
| butylated hydroxyanisole | antioxidant | 0.02% |
| trisodium citrate dihydrate | acidulant | 0.35% |
| TWEEN ® 80 | surfactant | 1.50% |
| (Polysorbate 80) | | |
| citric acid (anhydrous) | acidulant | 0.25% |
| sodium benzoate | preservative | 0.10% |
| disodium EDTA | chelating agent | 0.10% |
| Methyl paraben | preservative | 0.18% |
| Propyl paraben | preservative | 0.02% |
| Eucalyptol | cooling agent/ | 0.02% |
| | penetrating agent | |
| L-menthol crystals | cooling agent/ | 0.07% |
| | penetrating agent | |

As shown in Table 2, a preparation was made % with 1.5% w/w TWEEN® 80 and was initially clear, but became cloudy over time. Compared to the preparation of Table 1, the preparation of Table 2 was less sticky while drying on the skin.

### Example 3

TABLE 3
Exemplary hemorrhoid treatment preparation of this invention

| Ingredient | Ingredient Function | % w/w |
|---|---|---|
| water | solvent | 73.63% |
| glycerin | emollient | 20.00% |
| pramoxine hydrochloride | analgesic | 1.00% |
| phenylephrine hydrochloride | vasoconstrictor | 0.25% |
| propylene glycol | emollient | 2.00% |
| butylated hydroxyanisole | antioxidant | 0.05% |
| trisodium citrate dihydrate | acidulant | 0.35% |
| TWEEN ® 80 | surfactant | 2.00% |
| (Polysorbate 80) | | |
| citric acid (anhydrous) | acidulant | 0.25% |
| sodium benzoate | preservative | 0.10% |
| disodium EDTA | chelating agent | 0.10% |
| Methyl paraben | preservative | 0.18% |
| Propyl paraben | preservative | 0.02% |
| L-menthol crystals | cooling agent/ | 0.07% |
| | penetrating agent | |

As shown in Table 3, a preparation was made with 2.0% w/w TWEEN® 80 and was clear, and maintained clarity over the shelf life of two years. Compared to the preparation of Table 1, the preparation of Table 3 was less sticky while drying on the skin.

All publications (e.g. Non-Patent Literature), patents, patent application publications, and patent applications mentioned in this specification are indicative of the level of skill of those skilled in the art to which this invention pertains. All such publications (e.g. Non-Patent Literature), patents, patent application publications, and patent applications are herein incorporated by reference to the same extent as if each individual publication, patent, patent application publication, or patent application was specifically and individually indicated to be incorporated by reference.

While the foregoing invention has been described in connection with this preferred embodiment, it is not to be limited thereby but is to be limited solely by the scope of the claims which follow.

1. A composition for treating hemorrhoids, comprising:
an analgesic,
a vasoconstrictor,
an emollient, and
a cooling agent,
wherein the composition does not comprise a lipophilic skin protectant.

2. The composition for treating hemorrhoids of claim 1, wherein the analgesic comprises at least one member selected from the group consisting of benzocaine, benzyl alcohol, dibucaine, dibucaine hydrochloride, diclonine hydrochloride, lidocaine, pramoxine hydrochloride, tetracaine and tetracaine hydrochloride.

3. The composition for treating hemorrhoids of claim 1, wherein the analgesic is present in an amount of 0.25-20% by weight.

4. (canceled)

5. The composition for treating hemorrhoids of claim 1, wherein the vasoconstrictor comprises at least one member selected from the group consisting of ephedrine sulfate, epinephrine, epinephrine hydrochloride and phenylephrine hydrochloride.

6. The composition for treating hemorrhoids of claim 1, wherein the vasoconstrictor is present in an amount of 0.005-1.25% by weight.

7. (canceled)

8. The composition for treating hemorrhoids of claim 1, wherein the emollient comprises at least one member selected from the group consisting of dipropylene glycol, hexylene glycol, butylene glycol, propylene glycol and glycerin.

9. The composition for treating hemorrhoids of claim 1, wherein the emollient is present in an amount of 1.0-40% by weight.

10-11. (canceled)

12. The composition for treating hemorrhoids of claim 1, further comprising a solvent.

13-14. (canceled)

15. The composition for treating hemorrhoids of claim 1, further comprising a non-ionic surfactant.

16-30. (canceled)

31. The composition for treating hemorrhoids of claim 1, further comprising a solvent,
a non-ionic surfactant,
an acidulant,
an antioxidant,
a preservative, and
a chelating agent.

32. The composition for treating hemorrhoids of claim 31, wherein the analgesic is present in an amount of 0.25-20% by weight,
the vasoconstrictor is present in an amount of 0.005-1.25% by weight;
the emollient is present in an amount of 1.0-40% by weight,
the cooling agent is present in an amount of 0.05-0.1% by weight,
the solvent is present in an amount of 70-90% by weight,
the non-ionic surfactant is present in an amount of 0.5-5.0% by weight,
the acidulant is present in an amount of 0.10-0.50% by weight,
the antioxidant is present in an amount of 0.01-0.10% by weight,
the preservative is present in an amount of 0.01-0.25% by weight, and
the chelating agent is present in an amount of 0.05-0.20% by weight.

33-35. (canceled)

36. The composition for treating hemorrhoids of claim 1, wherein the composition is formulated as a member selected from the group consisting of a solution, a suppository, an ointment or an impregnated sheet.

37-39. (canceled)

40. The composition for treating hemorrhoids of claim 1, wherein the composition does not comprise petrolatum, witch hazel, cetyl alcohol, acyclic alcohols or monohydric aliphatic alcohols.

41. A sheet for treating hemorrhoids, wherein the sheet is impregnated with a composition comprising an analgesic,
a vasoconstrictor,
an emollient, and
a cooling agent.

42. (canceled)

43. The sheet for treating hemorrhoids of claim 41, wherein the sheet comprises at least one member selected from the group consisting of absorbent cellulose based fibers and absorbent synthetic fibers.

44-60. (canceled)

61. A method of making the composition for treating hemorrhoids of claim 31, comprising:
adding an antioxidant to a non-ionic surfactant and a first emollient in a first tank, to form a first mixture;
mixing the first mixture;
adding a first preservative and a cooling agent to the first mixture in the first tank, to form a second mixture;
mixing the second mixture;
adding a solvent to a second emollient, different from the first emollient, in a second tank, to form a third mixture;
adding a chelating agent, a second preservative, different from the first preservative, and an acidulant to the third mixture in the second tank, to form a fourth mixture;
mixing the fourth mixture;
adding an analgesic and a vasoconstrictor to the fourth mixture in the second tank, to form a fifth mixture;
mixing the fifth mixture;
adding the second mixture to the fifth mixture in the second tank, to form a final mixture;
mixing the final mixture to form the composition for treating hemorrhoids.

62. (canceled)

63. A method of treating hemorrhoids, comprising:
applying the composition for treating hemorrhoids of claim 1 to a hemorrhoid.

64-65. (canceled)

66. A composition for treating hemorrhoids, consisting of:
an analgesic,
a vasoconstrictor,
an emollient,
a cooling agent,
optionally, a solvent,
optionally, an acidulant,
optionally, an antioxidant,
optionally, a preservative, and
optionally, a chelating agent,
wherein the composition does not comprise a lipophilic skin protectant.

67. The composition for treating hemorrhoids of claim 31, wherein the analgesic comprises pramoxine hydrochloride,
the vasoconstrictor comprises phenylephrine hydrochloride;
the emollient comprises glycerin and propylene glycol,
the cooling agent comprises menthol,
the solvent comprises water,
the non-ionic surfactant comprises polysorbate 80,
the acidulant comprises sodium citrate and citric acid,
the antioxidant comprises butylated hydroxyanisole,
the preservative comprises sodium benzoate, methyl paraben and propyl paraben, and
the chelating agent comprises disodium EDTA.

68. (canceled)

69. A method of treating hemorrhoids, comprising:
applying the composition for treating hemorrhoids of claim 32 to a hemorrhoid.

70-71. (canceled)
